SEQUENCE LISTING (2 sequences)

SEQ ID NO: 1 - DNA, 1200 bp, Escherichia coli
  CDS (557)..(1171)
agaaataatgtggagatcgcaccgcccatcgaatgtgccagtatatagcgtttacgccacggaccgggctgaacctcctgctgccagaatgccgccagatcatcaacataatcattaaagcgattaacatgcccgagatgcggatcggctaacaggcgaccggaacgtccctgcccgcgatggtcgatgattaagacatcaaaccccaaatggaacaggtcataggccagttccgcatattttacgtagctctcaatacgccccgggcagatgactaccacccggtcatggtgctgtgcgcgaaaacggacaaagcgcaccggaatgtcatccacaccagtaaactctgcttcatcacgctgacgccagaaatcagtcagcggtcccatggtaaaagcagcaaacgcgttttctcttgtttcccagtctttttgctgctgaaacatcgggtaatctgcctcttaaaccacgtaaaatcgttttttttagcgtgcctgacacaacgctgcgacagtagcgtattgtggcacaaaaatagacacaccgggagttcatcatgaccttagaatggtggtttgcctacctgctgacatcgatcattttaacgctgtcgccaggctctggtgcaatcaacactatgaccacctcgctcaaccacggttatccggccggtggcgtctattgctgggcttcagaccggactggcgattcatattgtgctggttggcgtggggttgggacgctattttcccgctcagtgattgcgtttgaagtgttgaagtgggcaggcgcggcttacttgatttggctgggaatccagcagtggcgcgccgctggtgcaattgaccttaaatcgctggcctctactcaatcgcgtcgacatttgttccagcgcgcagtttttgtgaatctcaccaatcccaaaagtattgtgtttctggcggcgctatttccgcaattcatcatgccgcaacagccgcaactgatgcagtatatcgtgctcggcgtcaccactattgtggtcgatattattgtgatgatcggttacgccacccttgctcaacggattgctctatggattaaaggaccaaagcagatgaaggcgctgaataagattttcggctcgttgtttatgctggtgggagcgctgttagcatcggcgaggcatgcgtgaaaaataatgtcggatgcggcgtaaac

SEQ ID NO: 2 - protein, 205 aa, Escherichia coli
MTLEWWFAYLLTSIILTLSPGSGAINTMTTSLNHGYPAGGVYCWASDRTGDSYCAGWRGVGTLFSRSVIAFEVLKWAGAAYLIWLGIQQWRAAGAIDLKSLASTQSRRHLFQRAVFVNLTNPKSIVFLAALFPQFIMPQQPQLMQYIVLGVTTIVVDIIVMIGYATLAQRIALWIKGPKQMKALNKIFGSLFMLVGALLASARHA

(19) United States
(12) Patent Application Publication
(10) Pub. No.: US 2006/0040364 A1
(43) Pub. Date: Feb. 23, 2006

(54) DNA coding for a protein which imparts L-homoserine resistance to Escherichia coli bacterium, and a method for producing L-amino acids

(76) Inventors: Vitaly Arkadievich Livshits, Moscow (RU); Natalia Pavlovna Zakataeva, Moscow (RU); Vladimir Venyamiovich Aleshin, Kaluga region (RU); Alla Valentinovna Belareva, Moscow (RU); Irina Lvovna Tokmakova, Moscow region (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/091,899

(22) Filed: Mar. 29, 2005

Related U.S. Application Data
(63) Continuation-in-part of application No. 09/847,392, filed on May 3, 2001, now Pat. No. 6,887,691.
Application No. 09/847,392 is a continuation of application No. 09/396,357, filed on Sep. 15, 1999, now Pat. No. 6,303,348.

(30) Foreign Application Priority Data
Oct. 13, 1998 (RU) 98118425

(51) Int. Cl. C12P 13/04; C07H 21/04; C12N 15/74; C12N 1/21

(52) U.S. Cl. 435/106; 435/252.33; 435/473; 536/23.2

(57) ABSTRACT

A bacterium which has an ability to produce an amino acid and in which a novel rhtB gene encodes a protein having an activity of making a bacterium having the protein L-homoserine-resistant is enhanced, is cultivated in a culture medium to produce and cause accumulation of the amino acid in the medium, and the amino acid is recovered from the medium.

This application claims benefit as a continuation-in-part under 35 U.S.C. §120 to U.S. patent application Ser. No. 09/847,392, which is a continuation of Ser. No. 09/396,357, now U.S. Pat. No. 6,303,348.

### BACKGROUND OF THE INVENTION

1. Technical Field

The present invention relates to a method for producing an amino acid, and especially a method for producing L-homoserine, L-alanine, L-isoleucine, L-valine, or L-threonine using a bacterium belonging to the genus Escherichia.

2. Background Art

The present inventors obtained, with respect to E. coli K-12, a mutant having a mutation thrR (hereinafter, “rhtA23”) that results in high concentrations of threonine (>40 mg/ml) or homoserine (>5 mg/ml) in a minimal medium (Astaurova, O. B. et al., Appl. Bioch. and Microbiol., 21, 611-616 (1985)). On the basis of the rhtA23 mutation, an improved threonine-producing strain (SU patent No. 974817), and homoserine- and glutamic acid-producing strains (Astaurova et al., Appl. Boch. And Microbiol., 27, 556-561 (1991)) were obtained.

Furthermore, the present inventors have reported that the rhtA gene exists at 18 min on the E. coli chromosome, and is identical to the ORFI between pexB and ompX genes. DNA expressing a protein encoded by the ORFI has been designated the rhtA (rht: resistance to homoserine and threonine) gene. The rhtA gene includes a 5′-noncoding region, which includes a SD sequence, an ORFI, and a terminator. Also, the present inventors have found that a wild-type rhtA gene imparts resistance to threonine and homoserine if cloned in a multi-copy state, and that enhancement of expression of the rhtA gene improves amino acid productivity of a bacterium belonging to the genus Escherichia which has an ability to produce L-lysine, L-valine or L-threonine (ABSTRACTS of 17th International Congress of Biochemistry and Molecular Biology in conjugation with 1997 Annual Meeting of the American Society for Biochemistry and Molecular Biology, San Francisco, Calif. Aug. 24-29, 1997, abstract No. 457).

The present inventors have found during the cloning of the rhtA gene that at least two distinct genes which impart homoserine resistance in a multi-copy state exist in E. coli. One is the rhtA gene, and the other has not been previously reported.

### SUMMARY OF THE INVENTION

An object of the present invention is to provide a novel gene which imparts resistance to homoserine, and a method for producing an amino acid, especially, L-homoserine, L-alanine, L-isoleucine, L-valine, and L-threonine with a high yield.

The inventors have found that a region at 86 min on the E. coli chromosome, when cloned into a multi-copy vector, is able to impart resistance to L-homoserine to E. coli, and that when the region is amplified, the amino acid productivity of the E. coli is improved, similar to the rhtA gene. On the basis of these findings, the present invention has been completed.

It is an object of the present invention to provide a DNA coding for a protein selected from the group consisting of:
(A) a protein comprising an amino acid sequence shown in SEQ ID NO: 2; and
(B) a protein comprising an amino acid sequence which includes deletion, substitution, insertion, or addition of one or several amino acids in the amino acid sequence shown in SEQ ID NO: 2, and wherein said protein has an activity of making a bacterium having the protein L-homoserine-resistant,

It is an object of the present invention to provide the DNA as described above which is a DNA selected from the group consisting of:
(a) a DNA comprising a nucleotide sequence corresponding to numbers 557 to 1171 shown in SEQ ID NO: 1; and
(b) a DNA which is able to hybridize with the nucleotide sequence of numbers 557 to 1171 shown in SEQ ID NO: 1 under stringent conditions, and wherein said DNA codes for a protein having the activity of making the bacterium having the protein L-homoserine-resistant.

It is an object of the present invention to provide a bacterium belonging to the genus Escherichia, wherein L-homoserine resistance of the bacterium is enhanced by amplifying a copy number of the above-described DNA in the bacterium.

It is a further object of the present invention to provide the bacterium as described above wherein the above-described DNA is carried on a multicopy vector in the bacterium,

It is an object of the present invention to provide the bacterium as described above wherein the above-described DNA is carried on a transposon in the bacterium.

It is an object of the present invention to provide a method for producing an amino acid, comprising cultivating the bacterium as described above which has an ability to produce the amino acid, in a culture medium to produce and cause accumulatation of the amino acid in the medium, and recovering the amino acid from the medium.

It is an object of the present invention to provide the method as described above, wherein the amino acid is at least one selected from the group consisting of L-homoserine, L-alanine, L-isoleucine, L-valine, and L-threonine.

### BRIEF DESCRIPTION OF THE DRAWINGS

FIG. 1 shows the cloning, identification, and inactivation of the rhtB gene.

FIG. 2 shows the amino acid sequence of the RhtB protein.

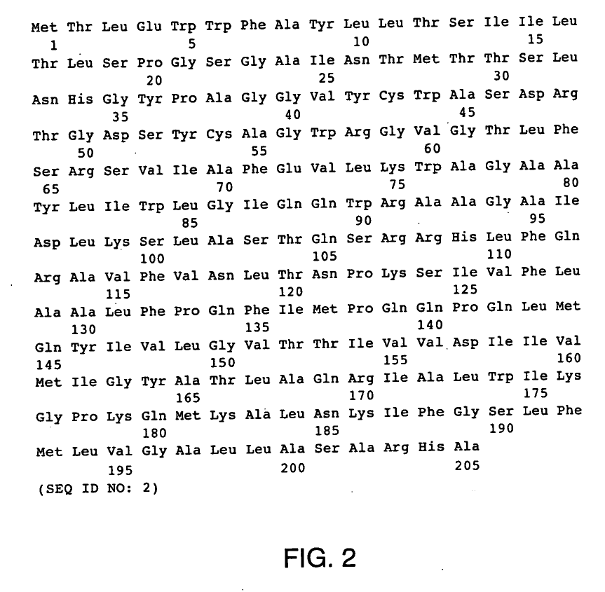

### DETAILED DESCRIPTION OF THE PREFERRED EMBODIMENTS

The DNA of the present invention may be referred to as the “rhtB gene.” A protein encoded by the rhtB gene may be referred to as the “RhtB protein.” An activity of the RhtB protein which imparts resistance to L-homoserine to a bacterium, i.e. an activity of making a bacterium having the RhtB protein L-homoserine-resistant, may be referred to as the “Rh activity.” A structural gene encoding the RhtB protein within the rhtB gene may be referred to as the “rhtB structural gene”. The phrase “enhancing the Rh activity” means imparting resistance to homoserine to a bacterium or enhancing the resistance by increasing the number of RhtB protein molecules, increasing a specific activity of the RhtB protein, or desensitizing negative regulation of the expression or the activity of the RhtB protein, or the like. The phrase “DNA coding for a protein” means a double-stranded DNA in which one strand codes for the protein. “L-homoserine resistance” means that a bacterium is able to grow on a minimal medium containing L-homoserine at a concentration at which a wild-type strain thereof cannot grow, usually 10 mg/ml. A bacterium having an “ability to produce an amino acid” means that the bacterium produces and is able to cause accumulation of larger amounts of an amino acid in a medium than a wild-type strain thereof.

According to the present invention, resistance to a high concentration of homoserine can be imparted to a bacterium belonging to the genus Escherichia. A bacterium belonging to the genus Escherichia, which has an increased resistance to homoserine and an ability to cause accumulation of an amino acid, especially, L-homoserine, L-alanine, L-isoleucine, L-valine, or L-threonine in a medium with a high yield is disclosed.

The present invention will be explained in detail below.

<1> DNA of the Present Invention

The DNA of the present invention encodes a protein which has Rh activity and an amino acid sequence of SEQ ID NO: 2. Specifically, the DNA of the present invention may be exemplified by a DNA comprising a nucleotide sequence of the numbers 557 to 1171 in SEQ ID NO: 1

The DNA of the present invention includes a DNA fragment encoding the RhtB protein which imparts resistance to homoserine to the bacterium Escherichia coli. The DNA of the present invention includes a DNA fragment which includes the regulatory elements of the rhtB gene and the structural part of rhtB gene, and which has the nucleotide sequence shown in SEQ ID NO: 1.

The nucleotide sequence shown in SEQ ID NO: 1 corresponds to a part of a sequence complementary to the sequence of GenBank accession number M87049. SEQ ID NO: 1 includes f138 (nucleotide numbers 61959-61543 of GenBank accession number M87049) which is a known ORF (open reading frame) located at 86 min on the E. coli chromosome, and 5′-flanking and 3′-flanking regions thereof, but the function of f138 is unknown. The f138, which contains only 160 nucleotides in the 5′-flanking region, cannot impart resistance to homoserine. No termination codon is present between the 62160 and 61959 of M87049 (upstream to the ORE f138). Hence, the coding region is 201 bp longer. Thus, the RhtB protein and the rhtB gene are novel.

The rhtB gene may be obtained, for example, by infecting Mucts lysogenic strain of E. coli using a lysate of a lysogenic strain of E. coli such as K12 or W3110 according to the method using mini-Mu d5005 phagemid (Groisman, E. A., et al., J. Bacteriol., 168, 357-364 (1986)), and isolating plasmid DNAs from colonies grown on a minimal medium containing kanamycin (40 μg/ml) and L-homoserine (10 mg/ml). As illustrated in the Example described below, the rhtB gene was mapped at 86 min on the chromosome of E. coli. Therefore, the DNA fragment including the rhtB gene may be obtained from the chromosome of E. coli by colony hybridization or PCR (polymerase chain reaction, refer to White, T. J. et al, Trends Genet. 5, 185 (1989)) using an oligonucleotide(s) which has a sequence corresponding to the region near the portion at 86 min on the chromosome of E. coli. Alternatively, the oligonucleotide may be designed according to the nucleotide sequence shown in SEQ ID NO: 1. By using oligonucleotides having nucleotide sequences corresponding to an upstream region from nucleotide number 557 and a downstream region from nucleotide number 1171 in SEQ ID NO: 1 as the primers for PCR, the entire coding region can be amplified.

Synthesis of the oligonucleotides can be performed by an ordinary method such as the phosphoamidite method (see Tetrahedron Letters, 22, 1859 (1981)) by using a commercially available DNA synthesizer (for example, DNA Synthesizer Model 380B produced by Applied Biosystems). Furthermore, PCR can be performed using a commercially available PCR apparatus (for example, DNA Thermal Cycler Model PJ2000 produced by Takara Shuzo Co., Ltd.), using Taq DNA polymerase (supplied by Takara Shuzo Co., Ltd.) in accordance with a method designated by the supplier.

The DNA of the present invention may encode a RhtB protein, or a variant thereof. Such a variant may include one or more deletions, substitutions, insertions, or additions of one or more amino acids at one or a plurality of positions, provided the Rh activity of RhtB protein encoded thereby is not disrupted. The number of changes in the variant protein depends on the position or the type of amino acid residues in the three dimensional structure of the protein. It may be 2 to 30, preferably 2 to 15, and more preferably 2 to 5 for the RhtB protein. These changes in the variants can occur in regions of the protein which are not critical for the function of the protein. This is because some amino acids have high homology to one another so the three dimensional structure or activity is not affected by such a change. These changes in the variant protein can occur in regions of the protein which are not critical for the function of the protein. The RhtB protein variant may be one which has a homology of not less than 50 to 70%, and more preferably 70% to 90%, and most preferably more than 95% with respect to the entire amino acid sequence of the RhtB protein shown in SEQ ID NO. 2, as long as the activity of RhtB protein is maintained. Homology between two amino acid sequences can be determined using the well-known methods, for example, the computer program BLAST 2.0, which calculates three parameters: score, identity and similarity.

The DNA which encodes the RhtB protein variant as described above, may be obtained, for example, by modifying the nucleotide sequence, for example, by means of the site-directed mutagenesis method so that one or more amino acid residues at a specified site is deleted, substituted, inserted, or added. DNA modified as described above may be obtained by conventionally known mutation treatments. These treatments includes treating DNA coding for the RhtB protein in vitro, for example, with hydroxylamine, and treating a microorganism, for example, a bacterium belonging to the genus Escherichia harboring a DNA coding for the RhtB protein with ultraviolet irradiation or a mutating agent such as N-methyl-N′-nitro-N-nitrosoguanidine (NTG) and nitrous acid, usually used for the mutation treatment.

The DNA which encodes the RhtB protein variant can be obtained by expressing a DNA which has been subjected to in vitro mutation treatment as described above in a multi-copy vector in an appropriate cell, investigating the resistance to homoserine, and selecting the DNA which imparts an increase in the resistance. Also, it is generally known that an amino acid sequence of a protein and a nucleotide sequence coding for it may be slightly different between species, strains, mutants, or variants, and therefore, the DNA which encodes an RhtB protein variant can be obtained from L-homoserine-resistant species, strains, mutants, and variants belonging to the genus Escherichia. Specifically, the DNA which encodes the RhtB protein variant can be obtained by isolating a DNA which is able to hybridize to a DNA having, for example, a nucleotide sequence of numbers 557 to 1171 shown in SEQ ID NO: 1 under stringent conditions, and which codes for a protein having Rh activity, from a bacterium belonging to the genus Escherichia which has been subjected to a mutation treatment, or a spontaneous mutant or variant of a bacterium belonging to the genus Escherichia. The term “stringent conditions” referred to herein are conditions under which a so-called specific hybrid is formed, and a non-specific hybrid is not formed. It is difficult to clearly express this condition by using any numerical value. However, for example, the stringent conditions include those under which DNAs having high homology to each other and are able to hybridize, for example, DNAs having homology of not less than 70%, preferably not less than 80%, more preferably not less than 90%, and most preferably not less than 95%, and DNAs having homology lower than the above to each other are not able to hybridize. Alternatively, the stringent conditions include those under which DNAs hybridize to each other at a salt concentration with washing typical of Southern hybridization, i.e., washing once or preferably 2-3 times under 1×SSC, 0.1% SDS at 60° C., preferably 0.1×SSC, 0.1% SDS at 60° C., more preferably 0.1×SSC, 0.1% SDS at 68° C.

<2> Bacterium Belonging to the Genus Escherichia of the Present Invention

The bacterium belonging the genus Escherichia of the present invention is a bacterium belonging to the genus Escherichia having enhanced Rh activity. A bacterium belonging to the genus Escherichia is exemplified by Escherichia coli. The Rh activity can be enhanced by, for example, amplifying the copy number of the rhtB structural gene in a cell, or transforming a bacterium belonging to the genus Escherichia with a recombinant DNA which includes the rhtB structural gene encoding the RhtB protein ligated to a promoter sequence which functions efficiently in a bacterium belonging to the genus Escherichia. The Rh activity can be also enhanced by substituting the promoter sequence of the rhtB gene on a chromosome with a promoter sequence which functions efficiently in a bacterium belonging to the genus Escherichia.

The amplification of the copy number of the rhtB structural gene in a cell can be performed by introducing a multi-copy vector which carries the rhtB structural gene into a bacterium belonging to the genus Escherichia. Specifically, the copy number can be increased by introduction of a plasmid, a phage, or a transposon (Berg, D. E. and Berg, C. M., Bio/Technol., 1, 417 (1983)) which carries the rhtB structural gene into a bacterium belonging to the genus Escherichia.

Examples of a multicopy vector include plasmid vectors such as pBR322, pMW118, pUC19, or the like, and phage vectors such as λ1059, λBF101, M13 mp9, or the like. The transposon is exemplified by Mu, Tn10, Tn5, or the like.

The introduction of a DNA into a bacterium belonging to the genus Escherichia can be performed, for example, by the method of D. M. Morrison (Methods in Enzymology 68, 326 (1979)), or a method in which recipient bacterial cells are treated with calcium chloride to increase the permeability of DNA (Mandel, M. and Higa, A., J. Mol. Biol., 53, 159 (1970)), and the like.

If the Rh activity is enhanced in an amino acid-producing bacterium belonging to the genus Escherichia as described above, the amount of amino acid produced can be increased. As the bacterium belonging to the genus Escherichia which is to have the Rh activity enhanced, strains which have abilities to produce desired amino acids are used. Alternatively, an ability to produce an amino acid may be imparted to a bacterium in which the Rh activity is enhanced. Examples of amino acid-producing bacteria belonging to the genus Escherichia are described below.

L-Threonine-Producing Bacteria

The L-threonine-producing bacteria belonging to the genus Escherichia may be exemplified by strain MG442 (Guayatiner et al., Genetika (in Russian), 14, 947-956 (1978)).

(2) L-Homoserine-Producing Bacteria

The L-homoserine-producing bacteria belonging to the genus Escherichia may be exemplified by strain NZ10 (thrB). This strain was derived from the known strain C600 (thrB, leuB) (Appleyard R. K., Genetics, 39, 440-452 (1954)) and is Leu⁺ revertant.

On the basis of the rhtB DNA fragment, new amino acid-producing strains E. coli NZ10/pAL4, pRhtB; E. coli MG422/pVIC40, pRhtB; and E. coli MG442/pRhtB were obtained which are useful for the production of amino acids by fermentation.

The new strains have been deposited in accordance with the Budapest Treaty in the Russian National Collection of Industrial Microorganisms (VKPM) on Oct. 6, 1998. The strain E. coli NZ10/pAL4, pRhtB was deposited and given accession number VKPM B-7658; the strain E. coli MG442/pRhtB was deposited and given accession number of VKPM B-7659; and the strain E. coli MG442/pVIC40,pRhtB was deposited and given accession number of VKPM B-7660.

The strain E. coli NZ10/pAL4, pRhtB (VKPM B-7658) exhibits the following culture, morphological, and biochemical features.

Cytomorphology: Gram-negative weakly-motile rods having rounded ends. Longitudinal size: 1.5 to 2 μm.

Culture Features:

Beef-extract agar—After 24-hour growth at 37° C., produces round whitish semitransparent colonies 1.5 to 3 mm in diameter, featuring a smooth surface, regular or slightly wavy edges, the center is slightly raised, homogeneous structure, pastelike consistency, readily emulsifiable.

Luria's agar—After a 24-hour growth at 37° C., develops whitish semitranslucent colonies 1.5 to 2.5 mm in diameter having a smooth surface, homogeneous structure, pastelike consistency, readily emulsifiable.

Minimal agar-doped medium M9—After 40 to 48 hours of growth at 37° C., forms colonies 0.5 to 1.5 mm in diameter, which are greyish-white in color, semitransparent, slightly convex, with a lustrous surface.

Growth in a beef-extract broth—After 24-hour growth at 37° C., exhibits strong uniform cloudiness, has a characteristic odor.

Physiological and Biochemical Features:

Grows upon thrust inoculation in a beef-extract agar. Exhibits good growth throughout the inoculated area. The microorganism proves to be a facultative anaerobe.

It does not liquefy gelatin.

Features good growth on milk, accompanied by milk coagulation.

Does not produce indole.

Temperature conditions—Grows on beef-extract broth at 20-42° C., an optimum temperature being within 33-37° C.

pH value of culture medium—Grows on liquid media having the pH value from 6 to 8, an optimum value being 7.2.

Carbon sources—Exhibits good growth on glucose, fructose, lactose, mannose, galactose, xylose, glycerol, and mannitol to produce an acid and gas.

Nitrogen sources—Assimilates nitrogen in the form of ammonium, nitric acid salts, as well as from some organic compounds.

Resistant to ampicillin, kanamycin and L-homoserine.

L-Threonine is used as a growth factor.

Content of plasmids—The cells contain the multi-copy hybrid plasmid pAL4 which ensures resistance to ampicillin and carries the thrA gene from the threonine operon, which codes for aspartate kinase-homoserine dehydrogenase I and is responsible for increased homoserine biosynthesis. Besides, the cells contain a multi-copy hybrid plasmid pRhtB which ensures resistance to kanamycin and carries the rhtB gene which confers resistance to homoserine (10 mg/l).

The strain E. coli MG442/pRhtB (VKPM B-7659) has the same culture, morphological, and biochemical features as the strain NZ10/pAL4, pRhtB, except that L-isoleucine is used as a growth factor instead of L-threonine. However, the strain can grow slowly without isoleucine. Besides, the cells of the strain contain only one multi-copy hybrid plasmid pRhtB which ensures resistance to kanamycin and carryies the rhtB gene which confers resistance to homoserine (10 mg/l).

The strain E. coli MG442/pVIC40,pRhtB (VKPM B-7660) has the same culture, morphological, and biochemical features as the strain NZ10/pAL4, pRhtB, except for L-isoleucine is used as a growth factor instead of L-threonine. However, the strain can grow slowly without isoleucine. The cells of the strain contain the multi-copy hybrid plasmid pVIC40 which ensures resistance to streptomycin and carryies the genes of the threonine operon. Besides, they contain multi-copy hybrid plasmid pRhtB which ensures resistance to kanamycin and carryies the rhtB gene which confers resistance to homoserine (10 mg/l).

<3> Method for Producing an Amino Acid

An amino acid can be efficiently produced by cultivating the bacterium having enhanced Rh activity by amplifying a copy number of the rhtB gene as described above, and which has an ability to produce the amino acid, in a culture medium, producing and causing accumulation of the amino acid in the medium, and recovering the amino acid from the medium. The amino acid is exemplified preferably by L-homoserine, L-alanine, L-isoleucine, L-valine, and L-threonine.

In the method of present invention, the cultivation of the bacterium belonging to the genus Escherichia, and the collection and purification of an amino acid from the liquid medium may be performed in a manner similar to conventional methods for producing an amino acid by fermentation using a bacterium. The cultivation medium may be either synthetic or natural, so long as the medium includes a carbon and nitrogen source and minerals and, if necessary, nutrients which the chosen bacterium requires for growth in appropriate amounts. The carbon source may include various carbohydrates such as glucose and sucrose, and various organic acids. Depending on the assimilatory ability of the chosen bacterium, alcohol including ethanol and glycerol may be used. As the nitrogen source, ammonia, various ammonium salts such as ammonium sulfate, other nitrogen compounds such as amines, a natural nitrogen source such as peptone, soybean hydrolyte, and digested fermentative microbe can be used. As minerals, monopotassium phosphate, magnesium sulfate, sodium chloride, ferrous sulfate, manganese sulfate, and calcium carbonate can be used.

The cultivation is preferably performed under aerobic conditions such as shaking, aeration, and stirring. The temperature of the culture is usually 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 3-day cultivation leads to the accumulation of the target amino acid in the medium.

Recovering the amino acid after cultivation can be performed by removing solids such as cells from the medium by centrifugation or membrane filtration, and then collecting and purifying the target amino acid by ion exchange, concentration, and crystalline fraction methods, and the like.

### EXAMPLES

The present invention will be more concretely explained below with reference to the following non-limiting Examples. In the Examples, an amino acid is of L-configuration unless otherwise noted.

### Example 1

### Obtaining the rhtB DNA Fragment

Cloning the rhtB Gene into Mini-Mu Phagemid

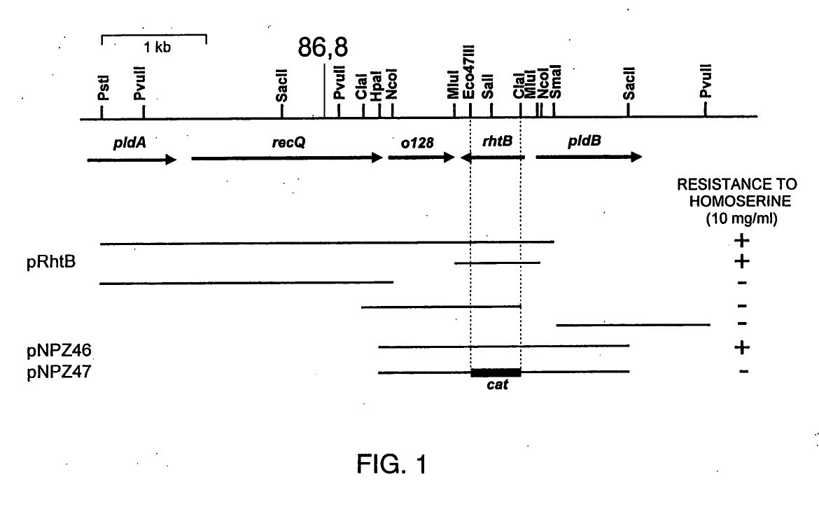

The wild-type rhtB gene was cloned in vivo using mini-Mu d5005 phagemid (Groisman, E. A., et al., J. Bacteriol., 168, 357-364 (1986)). MuCts62 lysogen of the strain MG442 was used as a donor. Freshly prepared lysates were used to infect a Mucts lysogenic derivative of a strain VKPM B-513 (Hfr K10 metB). The cells were plated on M9 glucose minimal medium with methionine (50 μg/ml), kanamycin (40 μg/ml) and homoserine (10 mg/ml). Colonies which appeared after 48 hr were picked and isolated. Plasmid DNA was isolated and used to transform the strain VKPM B-513 by standard techniques. Transformants were selected on L-broth agar plates with kanamycin as above. Plasmid DNA was isolated from those which were resistant to homoserine, and the inserted fragments were analyzed by restriction mapping. It appeared that two types of inserts belonging to different chromosome regions had been cloned from the donor. Thus, at least two different genes are present in multi-copy form and impart resistance to homoserine exist in E. coli. One of the two types of inserts is the rhtA gene which has already reported (ABSTRACTS of 17th International Congress of Biochemistry and Molecular Biology in conjugation with 1997 Annual Meeting of the American Society for Biochemistry and Molecular Biology, San Francisco, Calif. Aug. 24-29, 1997). The other type of insert is a fragment having a minimum length which imparts resistance to homoserine of 0.8 kb (FIG. 1).

(2) Identification of rhtB Gene

The insert fragment was sequenced by the dideoxy chain termination method of Sanger. Both DNA strands were sequenced in their entirety and all junctions were overlapped. The sequencing showed that the insert fragment included f138 (nucleotide numbers 61543 to 61959 of GenBank accession number M87049) which was a known ORF (open reading frame) present at 86 min of E. coli chromosome and 201 bp of an upstream region thereof (downstream region in the sequence of M87049), but the function is unknown. The f138 having only 160 nucleotides in the 5′-flanking region was not able to impart resistance to homoserine. No termination codon is present upstream the ORF f138 between 62160 and 61959 nucleotides of M87049. Furthermore, one ATG following a predicted ribosome binding site is present in the sequence. The larger ORF (nucleotide numbers 62160 to 61546) is designated as the rhtB gene. The RhtB protein deduced from the gene is highly hydrophobic and contains 5 possible transmembrane segments.

### Example 2

### Production of Homoserine-Producing Strain

Strain NZ10 of E. coli was transformed with the plasmid pAL4 to obtain the strain NZ10/pAL4. This plasmid was constructed by inserting a thrA gene, which encodes aspartokinase-homoserine dehydrogenase I, into a pBR322 vector. The strain NZ10 is a leuB⁺-reverted mutant (thrB) obtained from the E. coli strain C600 (thrB, leuB) (Appleyard, Genetics, 39, 440-452 (1954)).

The rhtB gene was inserted into plasmid pUK21, which is a known plasmid pUC19 having a kanamycin resistance gene substituted for an ampicillin resistance gene (Vieira, J. and Messing, J., Gene, 100, 189-194 (1991)), to obtain pRhtB.

The strain NZ10/pAL4 was transformed with pUK21 or pRhtB to obtain strains NZ10/pAL4, pUK21, and NZ10/pAL4, pRhtB.

The thus obtained transformants were each cultivated at 37° C. for 18 hours in a nutrient broth with 50 mg/l kanamycin and 100 mg/l ampicillin, and 0.3 ml of the obtained culture was inoculated into 3 ml of a fermentation medium having the following composition and containing 50 mg/l kanamycin and 100 mg/l ampicillin, in a 20×200 mm test tube, and cultivated at 37° C. for 46 hours with a rotary shaker. After cultivation, the amount of homoserine which accumulates in the medium, and the absorbance at 560 nm of the medium were determined by known methods.

Fermentation medium composition (g/L)

| | |
|---|---|
| Glucose | 80 |
| (NH₄)₂SO₄ | 22 |
| K₂HPO₄ | 2 |
| NaCl | 0.8 |
| MgSO₄ 7H₂O | 0.8 |
| FeSO₄ 7H₂O | 0.02 |
| MnSO₄ 5H₂O | 0.02 |
| Thiamine hydrochloride | 0.0002 |
| Yeast Extract | 1.0 |
| CaCO₃ | 30 |
| (CaCO₃ was separately sterilized.) | |

The results are shown in Table 1. The strain NZ10/pAL4, pRhtB was able to cause accumulation of a larger amount of homoserine than the strains NZ10/pAL4 and NZ10/pAL4, pUK21, which do not have an enhanced rhtB gene.

TABLE 1

| | | Accumulated amount of |
|---|---|---|
| Strain | OD₅₆₀ | homoserine (g/L) |
| NZ10/pAL4 | 16.4 | 3.1 |
| NZ10/pAL4, pUK21 | 14.3 | 3.3 |
| NZ10/pAL4, pRhtB | 15.6 | 6.4 |

### Example 3

### Production of Alanine, Valine, and Isoleucine Using a RhtB-Transformed Strain

E. coli strain MG442 is a known strain (Gusyatiner, et al., 1978, Genetika (in Russian), 14:947-956).

The strain MG442 was transformed with the plasmids pUK21 and pRhtB to obtain strains MG442/pUK21 and MG442/pRhtB.

The thus obtained transformants were each cultivated at 37° C. for 18 hours in a nutrient broth with 50 mg/l kanamycin, and 0.3 ml of the obtained culture was inoculated into 3 ml of the fermentation medium described in Example 3, which contains 50 mg/l kanamycin, in a 20×200 mm test tube, and cultivated at 37° C. for 40 hours with a rotary shaker. After cultivation, accumulated amounts of alanine, valine and isoleucine in the medium and an absorbance at 560 nm of the medium were determined by known methods.

The results are shown in Table 2. The strain MG442/pRhtB was able to cause accumulation of larger amounts of alanine, valine, and isoleucine than the strain MG442/pUK21, which does not have an enhanced rhtB gene.

TABLE 2

| | | Accumulated amount (g/L) | | |
|---|---|---|---|---|
| Strain | OD₅₆₀ | Alanine | Valine | Isoleucine |
| MG442/pUK21 | 13.4 | 0.2 | 0.2 | 0.3 |
| MG442/pRhtB | 13.7 | 0.7 | 0.5 | 0.5 |

### Example 4

### Production of Threonine-Producing Strain

The strain MG442 (Example 3) was transformed with the known plasmid pVIC40 (U.S. Pat. No. 5,175,107 (1992)) by an ordinary transformation method. Transformants were selected on LB agar plates containing 0.1 mg/ml streptomycin. Thus a novel strain MG422/pVIC40 was obtained.

The strain MG442/pVIC40 was transformed with pUK21 or pRhtB to obtain strains MG442/pVIC40, pUK21 and MG442/pVIC40, pRhtB.

The thus obtained transformants were each cultivated at 37° C. for 18 hours in a nutrient broth with 50 mg/l kanamycin and 100 mg/l streptomycin, and 0.3 ml of the obtained culture was inoculated into 3 ml of the fermentation medium described in Example 3, which contains 50 mg/l kanamycin and 100 mg/l streptomycin, in a 20×200 mm test tube, and cultivated at 37° C. for 46 hours with a rotary shaker. After cultivation, the accumulated amount of threonine in the medium, and an absorbance at 560 nm of the medium were determined by known methods.

The results are shown in Table 3. The strain MG442/pVIC40, pRhtB was able to cause accumulation of larger amounts of threonine than the strains MG442/pVIC40 and MG442/pVIC40,pUK21, which do not have an enhanced rhtB gene.

TABLE 3

| | | Accumulated amount of |
|---|---|---|
| Strain | OD₅₆₀ | threonine (g/L) |
| MG442/pVIC40 | 17 | 13.6 |
| MG442/pVIC40, pUK21 | 16.3 | 12.9 |
| MG442/pVIC40, pRhtB | 15.2 | 16.3 |

### Example 5

### Effect of RhtB Gene Inactivation and Amplification on Bacterium E. coli Resistance to Some Amino Acids and Amino Acid Analogues

To inactivate the chromosomal rhtB gene, the plasmid pNPZ46 was constructed (FIG. 1) on the basis of pUK21 vector. It harbors a DNA fragment from 86 min of E. coli chromosome, with the rhtB gene and 5′-flanking and 3′-flanking regions thereof. Then, the ClaI-Eco47III fragment of the pNPZ46 plasmid rhtB gene was substituted for an AsuII-BsrBI fragment containing a cat (CmR) gene from the pACYC184 plasmid (Chang and Cohen, J. Bacteriol., 134, 1141-1156, 1978), giving the pNPZ47 plasmid (FIG. 1). To introduce the obtained insertionally inactivated rhtB gene into the chromosome of the E. coli strain N99 (the streptomycin-resistant derivative of the known strain W3350 (Campbell, Virology, 14, 22-33, 1961)), the method of Parker and Marinus was used (Parker, B. and Marinus, M. G., Gene, 73, 531-535, 1988). The substitution of the wild-type allele for the inactivated one was accomplished by phage P1 transduction and Southern hybridization (Southern, E. M., J. Mol. Biol., 98, 503-517, 1975).

Then the susceptibility of the thus obtained E. coli strain N99 rhtB::cat, of the initial strain N99 (rhtB⁻), and of its derivative transformed with pRhtB plasmid, N99/pRhtB, to some amino acids and amino acid analogues was tested. Overnight cultures of the strains grown in M9 minimal medium at 37° C. with a rotary shaker (10⁹ cfu/ml) were diluted 1:100 and grown for 5 hours under the same conditions. Then the log phase cultures thus obtained were diluted and about 10⁴ live cells were applied to well-dried test plates with M9 agar containing doubling increments of amino acids or analogues. The minimum inhibitory concentration (MIC) of these compounds was examined after a 40-46 h cultivation. The results are shown in Table 4.

TABLE 4

| | MIC (μg/ml) | | |
|---|---|---|---|
| Substrate | N99 (rhtB⁺) | N99/pRhtB | N99 rhtB::cat |
| 1. L-homoserine | 250 | 30000 | 125 |
| 2. L-threonine | 30000 | 50000 | 30000 |
| 3. L-serine | 5000 | 10000 | 5000 |
| 4. L-valine | 0.5 | 1 | 0.5 |
| 5. AHVA | 50 | 2000 | 25 |
| 6. AEC | 10 | 25 | 10 |
| 7. 4-aza-DL-leucine | 40 | 100 | 40 |

It follows from Table 4 that multiple copies of rhtB imparted to cells increased resistance to threonine, serine, valine, α-amino-β-hydroxyvaleric-acid (AHVA), S-(2-aminoethyl)-L-cysteine (AEC), and 4-aza-DL-leucine. The inactivation of the rhtB gene, on the contrary, increased the cells' sensitivity to homoserine and AHVA. These results in conjunction with the data on homology of the RhtB protein to the LysE lysine efflux transporter of Corynebacterium glutamicum (Vrljic et al., Mol. Microbiol., 22, 815-826, 1996) indicate the presence of functional analogues for the rhtB gene product. The presumed efflux transporter, RhtB, has specificity to several substrates (amino acids), or may show non-specific effects as a result of amplification.

While the invention has been described in detail with reference to preferred embodiments thereof, it will be apparent to one skilled in the art that various changes can be made, and equivalents employed, without departing from the scope of the invention. The aforementioned documents, as well as the foreign priority document, RU98118425, and the parent application Ser. Nos. 09/847,392 and 09/396,357 which is now U.S. Pat. No. 6,303,348, are incorporated by reference herein in their entirety.

What is claimed is:

1. An isolated DNA encoding an RhtB protein selected from the group consisting of:
A) a protein comprising the amino acid sequence of SEQ ID NO: 2; and
B) a protein variant which is at least 80% homologous to the amino acid sequence of SEQ ID NO: 2 and imparts resistance to L-homoserine to a bacterium which expresses said protein variant.

2. The DNA of claim 1, wherein said variant is at least 90% homologous to the amino acid sequence of SEQ ID NO: 2.

3. The DNA of claim 1, wherein said variant is at least 95% homologous to the amino acid sequence of SEQ ID NO: 2.

4. An Escherichia bacterium which has been modified to amplify the expression of the DNA of claim 1 as compared to a non-modified bacterium.

5. The bacterium of claim 4, wherein said expression is amplified by increasing the copy number of said DNA.

6. The bacterium of claim 5, wherein said DNA is present on a multi-copy vector in said bacterium.

7. The bacterium of clam 5, wherein said DNA is present on a transposon in said bacterium.

8. A method for producing an amino acid comprising:
A) cultivating the bacterium of claim 4 in a medium, and
B) recovering said amino acid from the medium.

9. The method of claim 8, wherein said amino acid is selected from the group consisting of L-homoserine, L-alanine, L-isoleucine, L-valine, and L-threonine.
